(19) United States
(12) Patent Application Publication
(10) Pub. No.: US 2024/0042044 A1
(43) Pub. Date: Feb. 8, 2024

(54) POLYMER DRUGS FOR CANCER THERAPIES

(71) Applicant: VIRGINIA COMMONWEALTH UNIVERSITY, Richmond, VA (US)

(72) Inventors: Sandro DA ROCHA, RICHMOND, VA (US); Rodrigo S. HEYDER, RICHMOND, VA (US)

(21) Appl. No.: 17/641,994

(22) PCT Filed: Sep. 10, 2020
(86) PCT No.: PCT/US20/50050

§ 371 Date: Mar. 10, 2022

Related U.S. Application Data
(60) Provisional application No. 62/898,277, filed on Sep. 10, 2019.

(51) Int. Cl. A61K 47/54; A61K 47/69; A61K 47/59

(52) U.S. Cl. CPC A61K 47/554; A61K 47/6935; A61K 47/593; A61K 47/6953

(57) ABSTRACT

Polymeric drugs for disease therapy are provided. The repeat units of the polymers comprise at least one chemotherapeutic agent, e.g. a cancer drug. The linkages between repeat units are hydrolysable under physiological conditions, e.g. at targeted sites in vivo. Nanoparticles formed from the polymers are also provided.

### CROSS-REFERENCE TO RELATED APPLICATIONS

This application claims benefit of U.S. provisional patent application 62/898,277 filed Sep. 10, 2019.

### STATEMENT OF FEDERALLY SPONSORED RESEARCH AND DEVELOPMENT

This invention was made with government support under 1508363 awarded by National Science Foundation (NSF). The United States government has certain rights in the invention.

### BACKGROUND OF THE INVENTION

### Field of the Invention

The invention generally relates to improved polymer drugs for cancer therapy. In particular, the invention provides polymers whose repeat units comprise at least one chemotherapeutic agent, e.g. a cancer drug.

### Description of Related Art

Historically, many local diseases have been treated by systemic administration of drugs. In this approach, in order to achieve therapeutic levels of drugs at local disease sites, drugs are delivered (via oral administration or injection) at a high systemic concentration, often with adverse side effects. As an alternative, biocompatible synthetic crosslinked polymers may be used as carriers to encapsulate and deliver drugs to local sites within the body, thereby reducing the need for the systemic administration of high concentrations of drugs. However, such polymers also introduce unnecessary synthetic components into the body of a subject treated using such carriers.

Additional options for drug delivery are needed.

### SUMMARY OF THE INVENTION

Other features and advantages of the present invention will be set forth in the description of invention that follows, and in part will be apparent from the description or may be learned by practice of the invention. The invention will be realized and attained by the compositions and methods particularly pointed out in the written description and claims hereof.

The disclosure provides polymers whose repeat units are or comprise at least one chemotherapeutic agent. The linkages used to connect the repeat units are hydrolysable under physiological conditions. Thus, after administration of the polymers to a subject, active chemotherapeutic agents are released in situ at the location of hydrolysis, resulting in a higher local concentration of the active agent at the site of action. Nanoparticles formed from the polymers are also provided.

It is an object of this invention to provide a polymer comprising repeat units, at least a portion of which are chemotherapeutic agents linked by bonds that are hydrolysable under physiological conditions at a targeted location in vivo. In some aspects, the chemotherapeutic agents are anti-cancer agents. In further aspects, the anti-cancer agents are gemcitabine (GMT).

Also provided is a nanoparticle or fiber comprising a plurality of polymers comprising repeat units, at least a portion of which are chemotherapeutic agents linked by bonds that are hydrolysable under physiological conditions at a targeted location in vivo. In some aspects, the chemotherapeutic agents are anti-cancer agents. In further aspects, the anti-cancer agents are gemcitabine (GMT).

Also provided is a method of making chemotherapeutic polymers, comprising selecting chemotherapeutic monomers which have at least one chemically modifiable group; chemically modifying a plurality of the chemotherapeutic monomers by attaching, to the at least one chemically modifiable group, a cross-linkable functional group, to form cross-linkable chemotherapeutic monomers; and polymerizing the cross-linkable chemotherapeutic monomers to form the chemotherapeutic polymers.

Also provided is a method of treating cancer in a subject in need thereof, comprising administering to the subject a therapeutically effective amount of a composition comprising a plurality of the polymer of claim 2 or the nanoparticle of claim 5, and a physiologically compatible carrier.

### BRIEF DESCRIPTION OF THE DRAWINGS

FIG. 1. Schematic diagram of the synthesis and hydrolysis of the exemplary polymer polyGMT.

FIG. 2. Chemical Structure of Gemcitabine.

FIG. 3. Synthesis of diacid Gemcitabine.

FIG. 4. Acetylation of Gemcitabine diacid.

FIG. 5. Polymerization Reaction (Melting Condensation).

FIG. 6. SEM images of Poly-Gemcitabine.

FIG. 7. Accelerated hydrolysis of the (a) GMT monomer—GMT-2(CH₂) and (b) the GMT polymer-polyGMT-2(CH₂).

### DETAILED DESCRIPTION

Provided herein are polymers whose repeat units (monomers) are or comprise at least one chemotherapeutic agent, as well as nanoparticles made from the polymers. The linkages used to connect the repeat units within a polymer are hydrolysable under physiological conditions. Thus, after administration of the polymers or the nanoparticles to a subject, the repeat units which are active chemotherapeutic agents are released in situ at the location of hydrolysis. It is noted that the agents are not released as “pro-drugs” but rather as chemotherapeutic agents per se.

Synthesis of Chemotherapeutic Polymers

Chemotherapeutic Agents

A variety of chemotherapeutic agents (drugs) can be linked to form polymers as described herein. In general, chemotherapeutic agents that may be used in the practice of the invention contain one or more sterically accessible modifiable functional groups that can be modified by the covalent attachment of linkable (linking) groups. The attachment of one or more linking groups to a drug molecule renders that drug molecule capable of acting as a monomer or repeat unit that can be crosslinked to other monomers/repeat units to form a polymer.

Examples of modifiable functional groups to which linkers can be attached and that occur in chemotherapeutic agents include but are not limited to: OH, COOH, sulfate, phosphate, nitrate, amine, etc. The number of modifiable functionals groups per molecule or agent generally ranges from about 1-10 or more, such as 1, 2, 3, 4, 5, 6, 7, 8, 9, or 10, or more. However, the number is typically in the range of from about 1-5, such as about 1, 2, 3, 4, or 5. In some aspects, when multiple modifiable functional groups are present on a drug molecule, in the practice of the invention at least one is modified by covalent attachment of a linker, and more than one (e.g. all) or an intermediate number may be modified.

Examples of drugs that can be modified and polymerized as described herein include but are not limited to: gemcitabine (GMT), etc.

In some aspects, the chemotherapeutic agent is an anti-cancer agent such as gemcitabine (GMT).

Linking Groups

According to this disclosure, the modifiable functional groups of a molecule of a chemotherapeutic agent are modified by covalent attachment of a chemical group that is capable of forming linkages (linking group, linker, etc.), generally covalent linkages (covalent bonds), with other linking groups that are present on another molecule of a chemotherapeutic agent, which may be the same or different. Generally, the other linking groups are generally the same and they are generally present on another molecule of the same agent. However, the attachment of more than one type of linking group to an agent, or to a subset of a group of identical agents, is not precluded; and the attachment of identical or different linking groups to different agents is also not precluded. In the former case, some agents in a group of identical agents contain linking agents that differ from those of other agents in the group, so that linkages within the polymer that is formed differ, e.g. resulting in different hydrolysis rates. In the latter case, the resulting polymer comprises more than one type of chemotherapeutic agent, joined by the same or different linking groups. Generally, a plurality of only one type of agent are linked in a polymer using only one type of linker.

Examples of suitable linking groups include but are not limited to: aldehydes such as ethanedial, pyruvaldehyde, 2-formyl-malonaldehyde, glutaraldehyde, adipaldehyde, heptanedial, octanedial; di-glycidyl ether, diols such as 1,2-ethanediol, 1,3-propanediol, 1,4-butanediol, 2,3-butanediol, 1,5-pentanediol, benzene-1,4-diol, 1,6-hexanediol, tetra(ethylene glycol) diol), PEG, di-thiols such as 1,2-ethanedithiol, 1,3-propanedithiol, 1,4-butanedithiol, 2,3-butanedithiol, 1,5-pentanedithiol, benzene-1,4-dithiol, 1,6-hexanedithiol, tetra(ethylene glycol) dithiol), di-amine such as ethylene diamine, propane-1,2-diamine, propane-1,3-diamine, N-methylethylenediamine, N,N′-dimethylethylenediamine, pentane-1,5-diamine, hexane-1,6-diamine, spermine and spermidine, divinyladipate, divinylsebacate, diamine-terminated PEG, double-ester PEG-N-hydroxysuccinimide, and di-isocyanate-terminated PEG, epichlorohydrin, S-acetylthioglycolic acid N-hydroxysuccinimide ester, bromoacetic acid N-hydroxysuccinimide ester, N-(3-dimethylaminopropyl)-N′-ethylcarbodiimide, iodoacetic acid N-hydroxysuccinimide ester, 4-(N-maleimido)benzophenone 3-(2-pyridyldithio)propionic acid N-hydroxysuccinimide ester 3-maleimidobenzoic acid N-hydroxysuccinimide ester, N,N′-cystamine-bis-acrylamide, N,N′-methylene-bis-acrylamide, N,N′-ethylene-bis-acrylamide, etc.

Attachment of a linking group to a drug may take place in a single step or in multiple steps. For example, the modifiable functional group of the drug and/or the atom(s) by which the linker attaches to the modifiable functional group may first be modified or activated to facilitate attachment. Similarly, after attachment of the linker to the drug molecule, the linker may be modified to facilitate polymerization. For example, if succinate is used as the linker, the COOH groups may be acetylated to facilitate polymerization.

The pairing of a drug with a suitable linking group is made based on any of a variety of considerations. Of course, the linker must be capable of forming a covalent bond with the modifiable functional group to which it is to be attached. In addition, the chemistry that modifies the drug can be varied (e.g. different types of linkers can be used) so as to achieve different thermophysical properties of the polymeric drug that is formed. For example, linking groups can be longer than e.g. succinic acid, more branched, aromatic, instead of aliphatic, include different functionalities, etc. These properties are useful as they will impact among other attributes of the polymer or nanoparticle, the rate of erosion, the rate of hydrolysis, and thus the rate of release of drug when in contact with the targeted physiological environment, and the desired targeted location of hydrolysis. For example, if a drug is delivered orally, the linkage may be designed to deliver the drug early (e.g. within the throat), or later (e.g. in the stomach) or even later (e.g. in the colon). The delivery site may be determined e.g. by the local pH at the site, by enzymes present at the site, etc. Any possible pairing of drug and linker may be used, as long as the bond(s) formed by the linkers is/are physiologically degradable at the targeted site of action, and as long as the monomer that is released is physiologically active in the manner that is intended, e.g. it functions as an anti-disease agent, such as an anti-tumor agent, at that site.

Formation of Polymers and Nanoparticles

Drug molecules modified by the attachment of one or more linkers are joined via covalent bonds to other modified drug molecules to form polymers. As used herein a “polymer” is a substance that has a molecular structure consisting chiefly (e.g. more than about 90%) or entirely of a large number of similar or identical units (monomers, repeats units) covalently bonded together by physiologically hydrolyzable bonds. The polymers may be linear or branched in two or three dimensions, depending on the number of linking groups that are present and/or their position(s) on the modified drug molecules, and the number of potential linking groups that actually become linked to another molecule. “Branched polymers” are generally those having secondary polymer chains linked to a primary backbone, resulting in a variety of polymer architectures such as star, H-shaped, pom-pom, and comb-shaped polymers, or combinations of these. Branched polymers may also have irregular architectures. Further, all repeat units (individual drug molecules) in a polymer are not necessary linked the same way. For example, some with three possible sites where linking may occur may be linked at one site, some at two sites and others at all three sites within the polymer.

Drug molecules that have been modified to contain linking groups are polymerized using protocols commensurate with the chemistry of the linkers. For example, when succinate is used as the linking group, The conditions are generally as described in Example 1, e.g. drug molecules were modified through the chemical conjugation of succinic acid molecules to the hydroxyl groups of the molecule, leading to the dicarboxylic acid form of the drug, and the added —COOH groups served as linkers between drug molecules during a polymerization step in which one monomer is bonded to e.g. two other monomers. In addition, the polymerization step was facilitated by the acetylation of the carboxylic acid groups by reacting the carboxylated monomers with a large excess of acetic anhydride.

In some aspects, polymer synthesized as described herein (such as the exemplary polyGMT) are further processed to generate engineered materials such as nanoparticles. As used herein, a nanoparticle is an ultrafine particle that is from about 1 to about 500 nanometers (nm) in a least one dimension, e.g. in diameter or thickness. Nanoparticles may have any of a variety of morphologies, including but not limited to substantially spherical or ovoid; 2-dimensional fibers; sheets or films; etc.

Any suitable technique may be used to generate nanoparticles from the polymers. For example, a quantity of polymeric material that has been solubilized or which forms a colloidal mixture or a suspension in a suitable solvent can be dialyzed against a solvent with lesser affinity for the polymer, which tends to drive the polymers to aggregate into nanoparticles. Such systems will of course vary depending on the hydrophobicity/hydrophilicity of the polymer, which in turn depends on the hydrophobicity/hydrophilicity of the repeat units. Exemplary systems include but are not limited to: a polymer in DMF or another suitable solvent is dialyzed against distilled water; etc.

After formation, the polymers or the nanoparticles formed from particles are generally further processed e.g. to form a concentrate or a solid for formulation into a pharmaceutical composition. This may be done, for example, by freezing and lyophilization, and/or drying and/or evaporation to form a particulate solid such as a powder or a crystalline material. At any stage of processing, the solids may be purified e.g. by washing, size exclusion chromatography, etc. to remove unreacted monomers and/or other unwanted reactions components. In addition, the pH may be adjusted, liquids may be concentrated, and solids may be milled (e.g. ground) as necessary for use in making a pharmaceutically acceptable medicament. It is noted that such medicaments may comprise polymers, nanoparticles formed from polymers or a mixture of the two.

Compositions and Administration

The polymers and nanoparticles described herein are generally delivered (administered) in a pharmaceutical composition. Such pharmaceutical compositions generally comprise a plurality of at least one type of the disclosed polymeric drugs or nanoparticles, i.e. one or more than one (a plurality) of different types of substantially purified polymeric drugs or nanoparticles (e.g. 2 or more such as 2, 3, 4, 5, 6, 7, 8, 9, 10 or more) may be included in a single formulation. Accordingly, the present invention encompasses such formulations and compositions. The compositions generally include one or more substantially purified polymers or nanoparticles as described herein, and a pharmacologically suitable (physiologically compatible) carrier, which may be aqueous or oil-based. Carriers and the characteristics of carriers are selected to prevent hydrolysis of the bonds between monomeric units of the polymers prior to delivery to a subject. For example, the pH of a carrier can be adjusted to prevent hydrolysis. For example, if hydrolysis requires a pH below e.g. 5.0, the carrier may have a pH about e.g. 8.0.

In some aspects, such compositions are prepared as liquid solutions or suspensions, or as solid forms such as tablets, pills, powders and the like. Solid forms suitable for solution in, or suspension in, liquids prior to administration are also contemplated (e.g. lyophilized forms), as are emulsified preparations. In some aspects, the liquid formulations are aqueous or oil-based suspensions or solutions. In some aspects, the active ingredients are mixed with excipients which may or may not be inert and which are pharmaceutically acceptable and compatible with the active ingredients, e.g. pharmaceutically acceptable salts. Suitable excipients include, for example, water, saline, dextrose, glycerol, ethanol and the like, or combinations thereof. In addition, the composition may contain minor amounts of auxiliary substances such as wetting or emulsifying agents, pH buffering agents, preservatives, and the like. If it is desired to administer an oral form of the composition, various thickeners, flavorings, diluents, emulsifiers, dispersing aids or binders and the like are added. The composition of the present invention may contain any such additional ingredients so as to provide the composition in a form suitable for administration. The final amount of chemotherapeutic agent (in the form of polymers and/or nanoparticles) in the formulations varies but is generally from about 1-99%. Still other suitable formulations for use in the present invention are found, for example in Remington's Pharmaceutical Sciences, 22nd ed. (2012; eds. Allen, Adejarem Desselle and Felton).

Some examples of materials which can serve as pharmaceutically acceptable carriers include, but are not limited to, ion exchangers, alumina, aluminum stearate, lecithin, serum proteins (such as human serum albumin), buffer substances (such as Tween™ 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

“Pharmaceutically acceptable salts” refers to the relatively non-toxic, inorganic and organic acid addition salts, and base addition salts, of compounds of the present invention. These: salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-.beta.-hydroxynaphthoates, gentisates, isethionates, di-p-toluoyltartrates, methanesulfonates, ethanesulfonates, benzenesulfonates, p-toluenesulfonates, cyclohexylsulfamates and laurylsulfonate salts, and the like. See, for example S. M. Berge, et al., “Pharmaceutical Salts,” J. Pharm. Sci., 66, 1-19 (1977) which is incorporated herein by reference. Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like.

The compositions may be administered in vivo by any suitable route including but not limited to: inoculation or injection (e.g. intravenous, intraperitoneal, intramuscular, subcutaneous, intra-aural, intraarticular, intramammary, and the like), topical application (e.g. on areas such as eyes, skin, in ears) and by absorption through epithelial or mucocutaneous linings (e.g., nasal, oral, vaginal, rectal, gastrointestinal mucosa, and the like) are also used. Other suitable means include but are not limited to: inhalation (e.g. as a mist or spray), orally (e.g. as a pill, capsule, liquid, etc.), intravaginally, intranasally, rectally, as eye drops, etc. In preferred embodiments, the mode of administration is oral or by injection.

In addition, the compositions may be administered in conjunction with other treatment modalities such as substances that boost the immune system, various other chemotherapeutic agents, surgery, radiation therapy, pain medication and the like, depending on the disease that is treated.

Diseases that are treated as described herein include but are not limited to: cancer, including but not limited to: Acute Lymphoblastic Leukemia (ALL), Acute Myeloid Leukemia (AML), Adrenocortical Carcinoma, AIDS-Related Cancers, Kaposi Sarcoma, AIDS-Related Lymphoma, Primary CNS Lymphoma, Anal Cancer, Appendix Cancer, Astrocytomas, Atypical Teratoid/Rhabdoid Tumor, Central Nervous System, Basal Cell Carcinoma, Bile Duct Cancer, Bladder Cancer, Bone Cancer, Ewing Sarcoma Family of Tumors, Osteosarcoma and Malignant Fibrous Histiocytoma, Brain Stem Glioma, Brain Tumor (e.g. Astrocytomas, Brain and Spinal Cord Tumors, Brain Stem Glioma, Central Nervous System Atypical Teratoid/Rhabdoid Tumor, Central Nervous System Embryonal Tumors, Central Nervous System Germ Cell Tumors, Craniopharyngioma, Ependymoma), Breast Cancer, Bronchial Tumors, Burkitt Lymphoma, Carcinoid Tumor, Gastrointestinal, Cardiac (Heart) Tumors, Central Nervous System (e.g. Atypical Teratoid/Rhabdoid Tumors, Embryonal Tumors, Germ Cell Tumors, Lymphomas), Cervical Cancer, Childhood Cancers, Cholangiocarcinoma, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Eye Cancer, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST), Hairy Cell Leukemia, Head and Neck Cancer, Heart Cancer, Hepatocellular (Liver) Cancer, Histiocytosis, Langerhans Cell, Hodgkin Lymphom, Hypopharyngeal Cancer, Intraocular Melanoma, Islet Cell Tumors, Pancreatic Neuroendocrine Tumors, Kaposi Sarcoma, Kidney (Renal Cell, Wilms Tumor and Other Childhood Kidney Tumors), Langerhans Cell Histiocytosis, Laryngeal Cancer, Leukemia (Acute Lymphoblastic (ALL), Acute Myeloid (AML), Chronic Lymphocytic (CLL), Chronic Myelogenous (CML), Hairy Cell), Lip and Oral Cavity Cancer, Liver Cancer (Primary), Lung Cancer (Non-Small Cell, Small Cell), Lymphoma, Macroglobulinemia, Non-Hodgkin Lymphoma, Malignant Fibrous Histiocytoma of Bone and Osteosarcoma, Melanoma, Intraocular (Eye), Merkel Cell Carcinoma, Mesothelioma, Malignant, Metastatic Squamous Neck Cancer with Occult Primary Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic (CML), Myeloid Leukemia, Acute (AML), Myeloma, Multiple, Myeloproliferative Neoplasms, Chronic, Nasal Cavity and Paranasal Sinus Cancer, Nasopharyngeal Cancer, Neuroblastoma, Non-Hodgkin Lymphoma, Oral Cancer, Oral Cavity Cancer, Lip and Oropharyngeal Cancer, Osteosarcoma and Malignant Fibrous Histiocytoma of Bone, Ovarian Cancer, Epithelial, Germ Cell Tumor, Low Malignant Potential Tumor, Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Central Nervous System (CNS) Lymphoma, Primary Peritoneal Cancer, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (Ewing, Kaposi, Osteosarcoma, Rhabdomyosarcoma, Soft Tissue, Uterine), Sézary Syndrome, Skin Cancer, Small Intestine Cancer, Squamous Cell Carcinoma, Squamous Neck Cancer with Occult Primary, Metastatic Stomach (Gastric) Cancer, T-Cell Lymphoma, Cutaneous, Testicular Cancer, Throat Cancer, Thymoma and Thymic Carcinoma, Thyroid Cancer, Ureter and Renal Pelvis, Transitional Cell Cancer, Urethral Cancer, Uterine Cancer, Vaginal Cancer, Vulvar Cancer and Wilms Tumor.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference, and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.). . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### EXAMPLES

EXAMPLE 1. Preparation of polymerized gemcitabine (polyGMT)

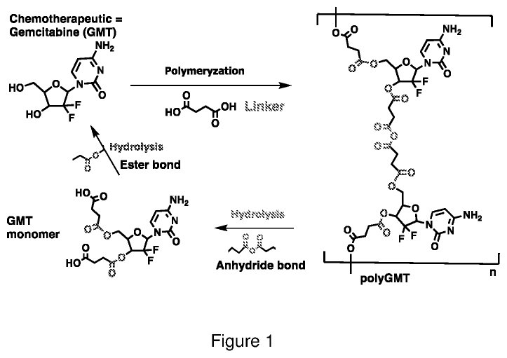

This Example describes the preparation of polyanhydride esters of gemcitabine (polyGMTs). The resulting polymers are hydrolysable under physiological conditions, thus leading to the release of the bioactive gemcitabine (GMT) itself (and not a pro-drug) as schematically illustrated in FIG. 1.

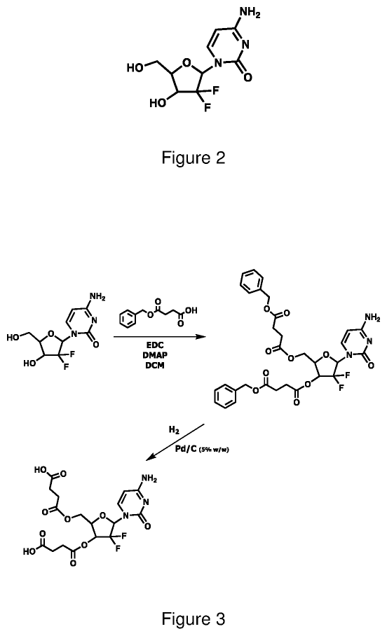

The synthesis of polyanhydrides requires the interconnection of two carboxylic acid groups (—COOH), which can be achieved using a number of different strategies. However, this group is absent on the gemcitabine molecule (FIG. 2). Thus, the GMT molecules were modified through the chemical conjugation of succinic acid molecules (FIG. 3) to the hydroxyl groups of the molecule, leading to the dicarboxylic acid form of GMT. The added —COOH groups served as linkers between gemcitabine molecules during a polymerization step in which one monomer is bonded to e.g. two other monomers. Ultimately, through repetitions in which the -COOH groups between monomers bond, a polymeric structure was formed.

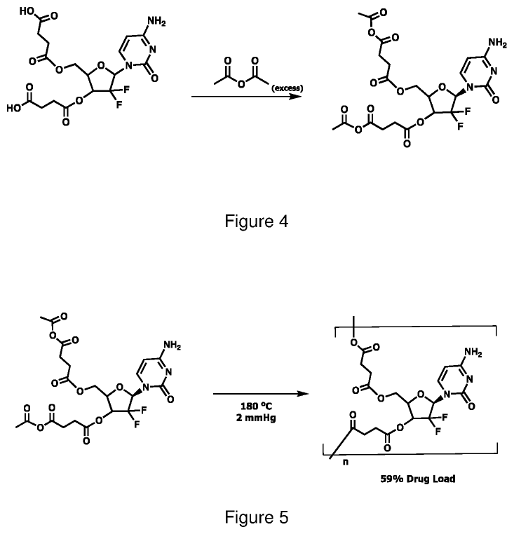

The polymerization step was facilitated by the acetylation of the carboxylic acid groups (FIG. 4). This was achieved by reacting the carboxylated monomers with a large excess of acetic anhydride. After the reaction, the reagent was removed under vacuum, leaving behind acetylated monomers. Acetylation of the monomers resulted in production of activated monomers (acetylated diacids) as well as some dimers and trimers. The monomers dimers and trimers were then submitted to a melting-condensation polymerization process (FIG. 5). In a reaction flask, the materials were submitted to high vacuum and high temperature and stirred by a mechanical overhead device. The monomer and intermediates were sampled and characterized through ¹H NMR, ¹³C NMR, and MALDI-ToF as the polymerization reaction to form progressed.

The reaction was maintained until the material, consistently showed the same viscosity (˜3h), indicating the presence of polymers. The polymers were then dissolved in dimethylformamide (DMF) and precipitated in an excess of diethyl ether. The solid precipitate was dried under vacuum, resulting in a dark colored solid and was characterized using gas phase chromatography (GPC), nuclear magnetic resonance (NMR) and differential scanning calorimetry (DSC). The characteristics of the polymer are shown in Table 1.

TABLE 1
Polymer Characterization

| | |
|---|---|
| Mw (Da) | 19,740 |
| Mn (Da) | 12,210 |
| PDI | 1.62 |
| Solvated Diameter (nm) | 1.4 |
| Tg | 124° C. |

EXAMPLE 2. Preparation of nanoparticles using polyGMT

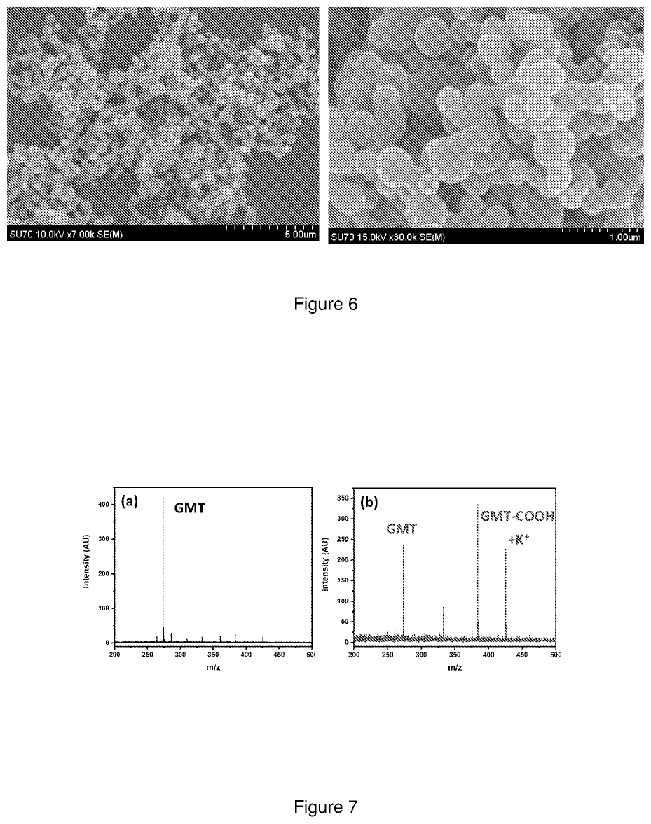

The synthesized polyGMT was further processed to generate engineered nanoparticles. Nanoparticles were formed by dialysis of a polymer solution against water. Briefly, 20 mg of polymer was solubilized in 2.5 mL of DMF and placed inside a dialysis membrane (MWCO 3,500). This bag was then immersed in 250 mL of distilled water and dialyzed for 6 hours; the aqueous phase was replaced after the first 3 hours. The bag's content was frozen and lyophilized resulting in a dark powder. The morphology and size of the particles was characterized by SEM and the results are shown in FIG. 6. The micrographs indicated nanoparticles having a spherical morphology and a diameter of 263±10 nm, as determined by ImageJ.

While the Invention has been described in terms of its several exemplary embodiments, those skilled in the art will recognize that the invention can be practiced with modification within the spirit and scope of the appended claims. Accordingly, the present invention should not be limited to the embodiments as described above but should further include all modifications and equivalents thereof within the spirit and scope of the description provided herein.

We claim:

1. A polymer comprising repeat units, at least a portion of which are chemotherapeutic agents linked by bonds that are hydrolysable under physiological conditions at a targeted location in vivo.

2. The polymer of claim 1, wherein the chemotherapeutic agents are anti-cancer agents.

3. The polymer of claim 2, wherein the anti-cancer agents are gemcitabine (GMT).

4. A nanoparticle or fiber comprising a plurality of polymers comprising repeat units, at least a portion of which are chemotherapeutic agents linked by bonds that are hydrolysable under physiological conditions at a targeted location in vivo.

5. The nanoparticle of claim 4, wherein the chemotherapeutic agents are anti-cancer agents.

6. The nanoparticle of claim 5, wherein the anti-cancer agents are gemcitabine (GMT).

7. A method of making chemotherapeutic polymers, comprising
selecting chemotherapeutic monomers which have at least one chemically modifiable group;
chemically modifying a plurality of the chemotherapeutic monomers by attaching, to the at least one chemically modifiable group, a cross-linkable functional group, to form cross-linkable chemotherapeutic monomers; and
polymerizing the cross-linkable chemotherapeutic monomers to form the chemotherapeutic polymers.

8. A method of treating cancer in a subject in need thereof, comprising
administering to the subject a therapeutically effective amount of a composition comprising
a plurality of the polymer of claim 2 or the nanoparticle of claim 5, and
a physiologically compatible carrier.
